(19) United States
(12) Patent Application Publication
(10) Pub. No.: US 2004/0258778 A1
(43) Pub. Date: Dec. 23, 2004

(54) Lactation cessation and breast engorgement compositions and method of use

(76) Inventors: Jill B. Farmar, Dallas, TX (US); Michael G. Farmar, Dallas, TX (US)

Correspondence Address: Charles S. Cotropia, Suite 3400, 717 N. Harwood, Dallas, TX, 75201, US

(21) Appl. No.: 10/814,607

(22) Filed: Mar. 31, 2004

Related U.S. Application Data
(60) Provisional application No. 60/459,099, filed on Mar. 31, 2003.

(51) Int. Cl. A61K 35/78

(52) U.S. Cl. 424/755; 424/758; 424/766

(57) ABSTRACT

A cabbage extract composition has now been found which is effective for accelerating lactation cessation in both breastfeeding and non-breastfeeding mothers. The cabbage extract composition can also be used to prevent or alleviate the symptoms of breast engorgement in lactating mothers.

### CROSS-REFERENCE TO RELATED APPLICATION

This application claims the benefit of U.S. Provisional Application No. 60/459,099 filed Mar. 31, 2003.

### TECHNICAL FIELD OF INVENTION

This invention relates to a bio-affecting and body-treating composition.

### BACKGROUND OF THE INVENTION

Breastfeeding, or nursing, has been identified as the ideal method of feeding and nurturing infants and is considered a primary factor in achieving optimal infant and child health, growth, and development. Epidemiologic research shows that human milk and breastfeeding of infants significantly decreases the risk for or severity of a large number of acute and chronic diseases such as diarrhea (Dewey, 1995; Beaudry, 1995), lower respiratory infection (Wright, 1989), otitis media (Duncan, 1993), bacteremia (Cochi, 1986), bacterial meningitis (Cochi, 1986), botulism (Arnon, 1984), urinary tract infection (Pisacane, 1992), and necrotizing enterocolitis (Lucas and Cole, 1990). There are also a number of studies that show a possible protective effect of human milk feeding against sudden infant death syndrome (Ford, 1993), insulin-dependent diabetes mellitus (Mayer, 1988), Crohn's disease (Koletzko, 1989), ulcerative colitis (Rigas, 1993), lymphoma (Shu, 1995), allergic diseases (Lucas, 1990), and other chronic digestive diseases (Greco, 1988). Breastfeeding has also been related to possible enhancement of cognitive development (Morrow-Tlucak, 1988).

There are also a number of studies that indicate possible health benefits for mothers. It has long been acknowledged that breastfeeding increases levels of oxytocin, resulting in less postpartum bleeding and more rapid uterine involution (Chua, 1994). Lactational amenorrhea causes less menstrual blood loss over the months after delivery. Recent research demonstrates that lactating women have an earlier return to pre-pregnant weight (Dewey, 1993), delayed resumption of ovulation with increased child spacing (Kennedy and Visness, 1992), improved bone remineralization postpartum (Melton, 1993) with reduction in hip fractures in the postmenopausal period (Cumming and Klineberg, 1993), and reduced risk of ovarian cancer (Rosenblatt and Thomas, 1993) and premenopausal breast cancer (Newcomb, 1994).

With these health benefits as well as social and economic advantages, the American Academy of Pediatrics and the World Health Organization have issued a policy statement that recommends breastfeeding exclusively for the first six months, then gradually introducing solid foods and continuing to breastfeed for at least six more months and after that for as long as mutually desired (American Academy of Pediatrics, 1997).

In normal breastfeeding practices, weaning an infant from breastfeeding to another food source such as solid food, formula or fruit juices is a gradual process. As the infant's diet incorporates other food sources, the mother continues to nurse but either decreases the amount of time at each feeding, decreases the number of breastfeedings each day and/or slowly increases the time between breastfeedings. For example, replacing a breastfeeding with a bottle or cup of fruit juice, formula, or cow's milk and increasing the number of substitutions per day over weeks and months has been recommended for easy weaning. Under these conditions, the mother's milk naturally diminishes at a slow rate as the infant's demand lessens.

There are circumstances, however, which either prevent a mother from breastfeeding or require that breastfeeding be discontinued abruptly. Some mothers prefer to bottle-feed rather than breastfeed. The death of an infant generally results in a mother's sudden halt to breastfeeding. Breastfeeding mothers who become pregnant may be advised to discontinue nursing, especially if the risk for miscarriage is high. Abrupt weaning is recommended for the infant with galactosemia (Rohr, 1985); the infant whose mother uses illegal drugs (American Academy of Pediatrics, Committee on Drugs, 1994); the infant whose mother has untreated active tuberculosis, and the infant whose mother has been infected with the human immunodeficiency virus (American Academy of Pediatrics, Committee on Pediatric Aids, 1995). Although most prescribed and over-the-counter medications are safe for the breastfed infant, many medications are not recommended including the following medications are contraindicated during breastfeeding: bromocriptine, cocaine, cyclophosphamide, cyclosporine, doxorubicin, ergotamine, lithium, methotrexate, phencyclidine, phenindione, radioactive iodine and other radiolabeled elements. Finally, there are some social considerations which may influence a mother's decision to discontinue breastfeeding such as a mother's return to work or an extended separation of mother and infant.

Whether a mother chooses not to breastfeed at all or to discontinue breastfeeding, lactation will continue for a time. If a mother does not nurse or empty her breasts postpartum, lactation naturally ceases in 1 to 2 weeks. Without the enhanced prolactin secretion caused by an infant's suckling during breastfeeding, the mother's milk production diminishes; however, milk production does not immediately cease. For women who are producing breast milk but not nursing, the mother can experience swollen, hard, painful breasts caused by milk stasis within the ductal network of the breasts. Also, plugged ducts and infection are common when a lactating mother abruptly weans. Thus, there is a need for products and methods to help mothers rapidly cease lactation.

Lactation can be suppressed by the administration of estrogens or diethylstilbestrol, which inhibit milk production by direct effects on the breast, or by administration of doparninergic agents such as bromocriptine which inhibit prolactin secretion by the pituitary. While estrogens have been used to treat painful breast engorgement, their limited large dose use for lactation cessation has been associated with stroke and blood-clot formation. According to the official labeling, up to 40 percent of the time, rebound engorgement occurs after a two-week course of treatment with bromocriptine ends. Further, very serious side effects have been reported during the use of bromocriptine to suppress lactation, including strokes, seizures (convulsions), intracranial bleeding, cerebral edema, and heart attacks (Iffy, 1998). Thus, pharmacological lactation cessation methods have been discouraged.

Other methods have been reported to treat the deleterious symptoms associated with lactation cessation. For example, administration of pain relievers such as acetaminophen or anti-inflammatory agents, ice or chilled gel packs, ultrasound treatment, and/or wearing a well-fitting bra or specially made breast binder have reportedly been used to treat the pain and discomfort experienced by mothers during abrupt weaning.

Use of cabbage leaf compresses to alleviate the swelling and pain associated with breast engorgement has also been previously reported (Corrieri, 1992; Rosier, 1988). For lactation cessation, the following method has been recommended: the large outer leaf of green cabbage is chilled and then crushed slightly or scored with a knife; one or more cold leaves are applied to each breast and secured in place with a bra; and the leaves are replaced with fresh cold leaves every two hours for about one week or until lactation cessation occurs. For breastfeeding mothers seeking temporary relief from the discomfort of engorgement, the cold leaves are applied for two to four hours. The use of cabbage leaf compresses has the advantages of being disposable, inexpensive and convenient; however, some individuals associate an unpleasant odor with their use and they have been reported to stain clothing.

The benefits of cabbage leaf compresses for breast engorgement have been evaluated by several groups with varying responses. Nikodem et al conducted a randomized, controlled study evaluating the effect of cabbage leaf application on mothers'perceptions of breast engorgement and the influence of the cabbage leaf treatment on breastfeeding practices, and reported that women who received the cabbage leaf application were more likely to breastfeed exclusively and for a longer period of time, but it was not clear whether the greater breastfeeding success might have been due to some beneficial effect of the cabbage leaf application or to secondary reassurance and improved confidence and self-esteem in the mothers (Nikodem, 1993). Snowden et al reviewed a variety of clinical trials for the treatment of breast engorgement and reported that (1) in three studies which used cabbage leaves or cabbage leaf extracts, no overall benefit was found and (2) since cabbage extract and placebo creams were equally effective, the alleviation in symptoms might have been brought about by other factors such as breast massage (Snowden, 2001). Roberts reported that mothers comparing the benefits of chilled cabbage leaves or a chilled gelpak found there was no difference in the degree of pain relief for the two treatments; however, the mothers preferred the cabbage leaf application (Roberts, 1995). In a study comparing the benefits of chilled vs. room temperature cabbage leaf applications in alleviating pain associated with breast engorgement, Roberts et al reported that mothers experienced significantly less pain with both chilled and room temperature cabbage leaf applications (Roberts et al. 1995). In a follow-up study, Roberts et al compared use of a cabbage leaf extract cream (1% cabbage extract prepared from common green cabbage by freeze-drying followed by extracting in alcohol) vs. a placebo cream for effectiveness in treating breast engorgement, and reported that the placebo group received equal relief to the treated group with the two groups showing no difference on all outcome measures, and with breastfeeding having a greater effect than the application of either cream on relieving discomfort and decreasing breast tissue hardness (Roberts, 1998).

A cabbage extract composition has now been found which is effective for accelerating lactation cessation in both breastfeeding and non-breastfeeding mothers and in alleviating the symptoms of breast engorgement.

### SUMMARY OF THE INVENTION

In one aspect, the invention is a method for treating a postpartum woman to encourage the cessation of lactation, comprising applying a composition comprising an amount of greater than 1% cabbage leaf extract by weight of the final composition to the affected area of the woman's breasts repeatedly each day, commencing on the date of delivery and extending until lactation subsides. In a preferred method, composition comprises an amount of about 1.1% to about 25% cabbage leaf extract by weight of the final composition. In a more preferred method, the composition comprises 5% cabbage leaf extract by weight of the final composition. In another preferred method, the composition comprises 5% cabbage leaf extract, 0.5% grape seed extract, and 0.5% cucumber extract by weight of the final composition. For all formulations, a preferred method further comprises the step of binding the breasts after the composition is applied to the breasts.

In another aspect, the invention is a method for treating a lactating woman to encourage the cessation of lactation, comprising applying a composition comprising an amount of greater than 1% cabbage leaf extract by weight of the final composition to the affected area of the woman's breasts repeatedly each day, commencing on a desired date and extending until lactation subsides. In a preferred method, the composition comprises an amount of about 1.1% to about 25% cabbage leaf extract by weight of the final composition. In a more preferred method, the composition comprises 5% cabbage leaf extract by weight of the final composition. In another preferred method, the composition comprises 5% cabbage leaf extract, 0.5% grape seed extract, and 0.5% cucumber extract by weight of the final composition. For all formulations, a preferred method further comprises binding the breasts after the composition is applied to the breasts.

In another aspect, the invention is a method for treating a lactating woman in need of treatment for or prevention of breast engorgement during lactation cessation, comprising applying a composition comprising an amount of greater than 1% cabbage leaf extract by weight of the final composition to the affected area of the woman's breasts repeatedly each day, commencing on a desired date and extending until lactation subsides. In a preferred method, the composition comprises an amount of about 1.1% to about 25% cabbage leaf extract by weight of the final composition. In a more preferred method, the composition comprises 5% cabbage leaf extract by weight of the final composition. In another preferred method, the composition comprises 5% cabbage leaf extract, 0.5% grape seed extract, and 0.5% cucumber extract by weight of the final composition. For all formulations, a preferred method further comprises binding the breasts after the composition is applied.

In another aspect, the invention is a method for treating a lactating woman for symptoms of breast engorgement, comprising applying one application of a composition comprising an amount of greater than 1% cabbage leaf extract by weight of the final composition to the affected area of the woman's breasts, commencing upon appearance of the symptoms. In a preferred method, the composition comprises an amount of about 1.1% to about 25% cabbage leaf extract by weight of the final composition. In a more preferred method, the composition comprises 5% cabbage leaf extract by weight of the final composition. In another preferred method, the composition comprises 5% cabbage leaf extract, 0.5% grape seed extract, and 0.5% cucumber extract by weight of the final composition. For all formulations, a preferred method further comprises applying the composition to the affected area of the woman's breasts once per day until symptoms subside.

In another aspect, the invention is a method for preventing symptoms of breast engorgement in a postpartum woman's breasts, comprising applying one application of a composition comprising an amount of greater than 1% cabbage leaf extract by weight of the final composition to the woman's breasts, commencing on the day milk comes in. In a preferred method, the composition comprises an amount of about 1.1% to about 25% cabbage leaf extract by weight of the final composition. In a more preferred method, the composition comprises 5% cabbage leaf extract by weight of the final composition. In another preferred method, the composition comprises 5% cabbage leaf extract, 0.5% grape seed extract, and 0.5% cucumber extract by weight of the final composition. For all formulations, a preferred method further comprises applying the composition to the woman's breasts once per day as symptoms appear.

In another aspect, the invention is a formulation for preventing or treating breast engorgement, comprising an extract of a vegetable leaf selected from the group consisting of Brassica oleracea capitata and Brassica Campestris Pekinenis in an amount comprising greater than 1% by weight of the formulation and an acceptable topical carrier. In a preferred formulation, the formulation comprises an amount of about 1.1% to about 25% cabbage leaf extract by weight of the final formulation. In a more preferred formulation, the formulation comprises 5% cabbage leaf extract by weight of the final formulation. In another preferred formulation, the formulation comprises 5% cabbage leaf extract, 0.5% grape seed extract, and 0.5% cucumber extract by weight of the final formulation.

In another aspect, the invention is a formulation for promoting lactation cessation in a lactating woman, comprising an extract of a vegetable leaf selected from the group consisting of Brassica oleracea capitata and Brassica Campestris Pekinenis in an amount comprising greater than 1% by weight of the formulation and an acceptable topical carrier. In a preferred formulation, the formulation comprises an amount of about 1.1% to about 25% cabbage leaf extract by weight of the final formulation. In a more preferred formulation, the formulation comprises 5% cabbage leaf extract by weight of the final formulation. In another preferred formulation, the formulation comprises 5% cabbage leaf extract, 0.5% grape seed extract, and 0.5% cucumber extract by weight of the final formulation.

In another aspect, the invention is a kit for the treatment of breast engorgement, comprising a formulation consisting of a cabbage extract and an acceptable topical carrier, the cabbage extract comprising greater than 1% by weight of the formulation, and instructions for the application of the formulation to a lactating woman. In a preferred configuration, the kit further comprises a breast-binding means selected from the group consisting of a tight-fitting garment or bandaging material.

In another aspect, the invention is a kit for promoting lactation cessation in a lactating woman, comprising a formulation consisting of a cabbage extract and an acceptable topical carrier, the cabbage extract comprising greater than 1% by weight of the formulation, and instructions for the application of the formulation to the lactating woman. In a preferred configuration, the kit further comprises a breast-binding means selected from the group consisting of a tight-fitting garment or bandaging material.

### DETAILED DESCRIPTION

In one aspect, the present invention includes compositions useful for promoting or accelerating lactation cessation. These compositions can be used by a non-breastfeeding mother who does not want her breast milk to come in, a breastfeeding mother interested in weaning an infant and seeking to stop breast milk production, and a lactating mother in need of treatment for the pain or discomfort associated therewith.

In another aspect, the present invention includes a method for promoting lactation cessation which includes the step of applying to the skin an effective amount of the cabbage extract composition as disclosed herein. For a postpartum non-breastfeeding mother, the composition is applied to the affected area of the breast repeatedly each day, preferably three to four times daily, for 7-10 days or until lactation subsides. When weaning an infant or child, a breastfeeding mother applies the composition to the entire breast area repeatedly each day, preferably three to four times daily, for 2-4 days or until lactation subsides. In conjunction with application of the composition, it is preferable for the subject to wear a tight-fitting garment such as a sports bra or elastic bandage covering the breasts to promote lactation cessation. Thus, in promoting lactation cessation, the method of the present invention comprises applying the cabbage extract composition frequently to the entire breast area (3 to 4 times daily) and binding the breasts with a tight-fitting garment.

For temporary relief of breast engorgement pain without diminishing milk production, the cabbage extract composition of the present invention is applied once to the affected area of the breast. In this case, the breasts are not bound in a tight-fitting garment after application of the composition. Changes in the breastfeeding schedule or skipping a feeding can result in the return of the engorgement pain or discomfort. If engorgement pain or discomfort returns or persists, then the cabbage extract composition can be applied to the affected area once a day as needed. However, a lactating mother may risk having her milk dry up altogether by such frequent application.

The cabbage extract composition of the present invention can also be administered to prevent the symptoms of breast engorgement in a postpartum woman at the initiation of milk production. The cabbage extract composition of the present invention is applied once to the affected area of the breast at the initiation of milk production. In this case, the breasts are not bound in a tight-fitting garment after application of the composition. If engorgement pain or discomfort begins, then the cabbage extract composition can be applied to the affected area once a day as needed. However, a lactating mother may risk having her milk dry up altogether by such frequent application.

For the purpose of promoting or accelerating lactation cessation, the composition of the present invention is applied to both breasts. The affected area of the breast to which the composition is applied includes the entire breast, i.e., nipple area and all breast tissue located radially outwardly therefrom.

For temporary relief of breast engorgement, a lactating mother can apply the composition of the present invention to one or both breasts as needed according to symptoms, with the affected area including the entire breast as defined above. It is understood that for localized pain caused by breast engorgement, application of the cabbage extract composition can be limited to the symptomatic portion of the breast.

The compositions of the preferred mode of the present invention comprise an extract of Brassica oleracea capitata, the green cabbage routinely used as a vegetable. There are a range of varieties useful in the present invention, including but not limited to Drumhead, Savoy, Danish, Domestic, Pointed, Jingan, Derby Day (Golden Acre), Charmant, Ruby Ball, Julius, Red Rodan, Danish Ballhead, Gloria, Bently, Rougette, January King, Savonarch and Wivoy. Red cabbage extract or extract of Brassica Campestris Pekinensis (Chinese cabbage) can also be used. The cabbage extract can be obtained from cabbage leaves using any extraction process known in the art for vegetable or botanical extraction, provided it meets the following physical parameters: pH 4.5-6.0; specific gravity 1.02-1.05 at 25° C.; light to medium yellow coloration; and preferably a maximum microbial count of less than 100 microorganisms per gram. Preferably, the cabbage extract is prepared using a cold process extraction method, e.g., extraction methods commonly practiced by European botanical extract companies. In the cold process extraction method, the cabbage is quick frozen soon after harvesting to temperatures far below the freezing point, thus preserving the chemical potency of the cabbage; while frozen, the cabbage leaves are pulverized in a solvent, preferably a recirculating hydroglycolic menstruum; and then the solvent is removed, leaving a cabbage extract in which the benefits of the cabbage leaf has been preserved. Other acceptable methods include a press process or solvent extraction process with the most common solvent being water. A combination of a press process with a solvent extraction process can also be used. Preferably, the press process and solvent extraction process are performed at temperatures at or below room temperature. If higher temperatures are used, the concentration of cabbage extract in the final composition may need to be increased for equivalent effectiveness. The cabbage extract can be immediately formulated into a cabbage extract composition or stored at room temperature for up to two years.

The cabbage extract composition of the present invention comprises cabbage extract and an acceptable topical carrier. In another preferred formulation, the composition comprises cabbage extract, grape seed extract (Vitis venifera), and cucumber extract (Cucumis sativus) in an acceptable topical carrier. A preferred composition of the present invention comprises greater than 1% cabbage extract by weight of the final formulation in an acceptable carrier for topical administration. A more preferred composition of the present invention comprises about 1.1% to about 25% cabbage extract by weight of the final formulation in an acceptable carrier for topical administration. An even more preferred composition of the present invention comprises 5% cabbage extract by weight of the final formulation in an acceptable carrier for topical administration. In another preferred composition, the cabbage extract composition of the present invention comprises about 1.1% to about 25% cabbage extract by weight of the final formulation, about 0.1% to about 1% grape seed extract by weight of the final formulation, about 0.1% to about 1.0% cucumber extract by weight of the final formulation and an acceptable topical carrier. A more preferred composition of the present invention comprises 5% cabbage extract by weight of the final formulation, 0.5% grape seed extract by weight of the final formulation, and 0.5% cucumber extract by weight of the final formulation in an acceptable carrier for topical administration.

The cabbage extract compositions of the present invention can be prepared in any compatible cream, gel, lotion, liquid or ointment preparation which can be safely topically applied to human skin. Tables I-VI provide exemplary formulations useful in compositions of the present invention. In each formulation, the cabbage extract can be added either in an aqueous phase, an oil phase, or after an aqueous phase is mixed with an oil phase.

In general, the cabbage extract compositions of the present invention will include water, one or more emulsifiers, one or more thickeners, one or more preservatives, preferably one or more humectants, one or more skin conditioning agents, and optionally, one or more emollients, one or more fragrances, and one or more coloring agents. The cabbage extract is preferably added to a base such as an oil-in-water emulsion, a water-in-oil emulsion, cream base or oil base. The formulation can be a semi-solid cream-like consistency, a lotion consistency, or an ointment consistency which can be packaged in a squeeze-type container, or a liquid consistency which can be packaged in a bottle with a flow-type cap or pump-type dispenser.

Whether the composition has a cream, gel, lotion, liquid or ointment base, the cabbage extract is added at greater than 1% to about 25% by weight, and preferably at 5% by weight of the final formulation. If present, grape seed extract and cucumber extract are added at about 0.1% to about 1.0% by weight of the final formulation. Preferably, the composition comprises water at about 50% to about 90%; one or more humectants at about 1% to about 10%; one or more emulsifiers at about 1% to about 30%; one or more thickeners at about 0.05% to about 30%; one or more skin conditioning agents at about 0.05% to about 2% and one or more preservatives at about 0.1% to about 1% (all percentages by weight of final formulation). When present, the composition comprises one or more emollients at about 1% to about 30%; one or more chelating agents at about 0.01% to about 1%; and one or more fragrances at less than about 1% (all percentages by weight of final formulation). The composition can also include a pH adjusting agent as needed as well as an antioxidant mixture, vitamins and/or anti-inflammatory agents. A preferred pH range for the composition is pH 6 to pH 7.

Suitable emulsifiers include but are not limited to cetyl alcohol, ceteareth-6, stearyl alcohol, ceteareth-25, polyethylene glycol 20 sorbitan monopalmitate, polyethylene glycol 5 soya sterol, sorbitan tristearate, sorbitan trioleate, glyceryl monopalmitate, diethanolamine cetyl phosphate, glyceryl monostearate, polyethylene glycol 100 stearate, polyethylene glycol 20 stearyl ether, polyethylene glycol ether of lauryl alcohol, polysorbate 80, lecithin and others approved for topical or cosmetic use.

Suitable thickeners include but are not limited to glyceryl stearate, stearic acid, caprylic/capric triglyceride, caprylic/capric triglyceride-sodium acrylate copolymer, fatty alcohols such as cetyl alcohol and stearyl alcohol, magnesium aluminum silicate, stearoxydimethicone, hydroxyethyl cellulose, propylene glycol monostearate, hydroxypropyl cellulose, carboxymethyl cellulose, xanthan gum, myristyl stearate, cetyl stearate, carbomers and others approved for topical or cosmetic use. For carbomers, triethanolamine is a suitable organic base.

Suitable emollients include but are not limited to butylene glycol, cetearyl octanoate, glyceryl stearate, stearic acid, corn oil, caprylic/capric triglyceride, caprylic/capric triglyceride-sodium acrylate copolymer, cetyl alcohol, dimethicone, mineral oil, petrolatum, glyceryl monooleate, myristyl alcohol, isopropyl palmitate, avocado oil, squalane, octyl palmitate, cocoa butter, sesame oil, propylene glycol dicaprylate/dicaprate, isopropyl myristate, diisopropyl dimerate, stearoxydimethicone and others approved for topical or cosmetic use.

Suitable moisturizers include but are not limited to glycerine, sodium pyrollidone carboxylic acid, d-panthenol, allantoin, tocopherol, tocopherol acetate, hydrolyzed animal protein and others approved for topical or cosmetic use.

Suitable humectants include but are not limited to butylene glycol, dimethicone, propylene glycol and others approved for topical or cosmetic use.

Suitable preservatives include imidazolidinyl urea, diazolidinyl urea, methylparaben, propylparaben, quaternium-15, dimethyldimethyl hydantoin, benzyl alcohol, phenoxyethanol and others approved for topical or cosmetic use.

TABLE I
Enriched Daily Lotion^{a,b}

| Ingredients | % Weight |
|---|---|
| Phase A: | |
| Deionized water | 79.05 |
| Butylene glycol | 5.00 |
| Phase B: | |
| Cremophor A6^{c} | 2.00 |
| Cremophor A25^{d} | 2.00 |
| Cetearyl alcohol | 3.00 |
| Luvitol EHO^{e} | 5.00 |
| Phase C: | |
| Luvigel EM^{f} | 0.40 |
| Phase D: | |
| Deionized water | 1.50 |
| Disodium EDTA | 0.20 |
| Sodium ascorbyl phosphates^{g} | 0.50 |
| D-panthenol 75 W^{h} | 0.50 |
| Phase E: | |
| Vitamin E acetate USPⁱ | 0.50 |
| Phase F: | |
| Preservative | 0.30 |
| Phase G: | |
| Fragrance | 0.05 |

aPublished at http://www.happi.com/current/Nov02formula.htm; Household and Personal Products Industry, 70 Hilltop Rd., Ramsey NJ 07446).
^{b}Procedure: while slowly mixing, combine Phase A ingredients; and heat to 75-80° C. In separate vessel, combine Phase B ingredients; heat to 75-80° C.; and mix with slow agitation. Add Phase C to Phase B; and mix until uniform. Transfer to sweep mixing and begin to cool batch to 45° C. Add pre-mixed Phase D. Add Phase E, Phase F and Phase G in order, and mix until uniform. Cool to 30-35° C.
^{c}BASF Corporation, Mount Olive, NJ; ceteareth-6 and stearyl alcohol.
^{d}BASF Corporation; ceteareth-25.
^{e}BASF Corporation; cetearyl octanoate.
^{f}BASF Corporation; caprylic/capric triglyceride sodium acrylate copolymer.
^{g}BASF Corporation.
^{h}BASF Corporation.
ⁱBASF Corporation; tocopheryl acetate.

TABLE II
Skin Treatment Lotion^{a,b}

| Ingredients: | % Wt. |
|---|---|
| Phase A | |
| Deionized water | 61.7 |
| Keltrol CG (xanthan gum)^{c} | 0.2 |
| Glycerin 99%^{d} | 5.0 |
| Multifruit BSC^{e} | 3.0 |
| Jeescreen Benzophenone-4^{d} | 0.1 |
| Jeechem GMS-165 (glyceryl stearate (and) PEG-100 stearate)^{d} | 3.0 |
| Phase B | |
| Jeesilc IDD (dimethicone crosspolymer-3 (and) isododecane)^{d} | 4.0 |
| Jeesilc 245 (cyclomethicone)^{d} | 8.0 |
| Jeesilc 200 MV (100 cst) (dimethicone)^{d} | 2.0 |
| Simugel NS^{f} | 4.0 |
| Phase C | |
| Jeesilc 6056 (dimethylpolysiloxane gum)^{d} | 3.0 |
| Jeecide G-II (propylene glycol (and) diazolidinyl urea | 1.0 |
| (and) methylparaben (and) propylparaben)^{d} | |
| Arnica Extract (arnica montana) | 2.0 |
| Flamingo Super Red | 1.0 |
| Phase D | |
| Jeesorb L-20 (polysorbate 20)^{d} | 1.0 |
| Vitamin E Acetate (tocopheryl acetate)^{d} | 0.5 |
| Fragrance | 0.5 |
| ^{a}Published at http://www.happi.com/current/Feb03formula.htm; Household and Personal Products Industry, 70 Hilltop Rd., Ramsey NJ 07446. | |
| ^{b}Procedure: Heat water to 65° C. Pre-mix Keltrol and glycerin and add to the water phase. Mix until dissolved. Add the other ingredients of phase A one at a time and mix well. Cool to 50° C. In the oil phase tank, add the Jeesilc IDD, Jeesilc 245 and Jeesilc 200 MV (100 cst) and mix until uniform. Add the Simugel and mix to 50° C. Using a homogenizer, add phase B to phase A and mix for 10 minutes. Cool to | |
| #40° C. Switch to prop agitation. Add the ingredients of phase C one at a time into the main tank and mix well after each addition. Pre-mix phase D in a side vessel and add to the main tank. Mix well. | |

^{c}C.P. Kelco U.S. Inc., 1313 N. Market St., Wilmington, DE 19894.
^{d}Arch Personal Care, 70 Tyler Place, South Plainfield, NJ 07080.
^{e}Jeen International Corp., 24 Madison Rd., Fairfield, NJ 07004.
^{f}Seppic, Inc., 30 Two Bridges Rd., Suite 210, Fairfield, NJ 07004.

TABLE III
Moisturizing Body Care Creme^{a,b}

| Ingredients: | % Wt. |
|---|---|
| Phase A | |
| Cremophor A6^{c} (ceteareth-6) | 2.0 |
| Cremophor A25^{c} (ceteareth-25) | 2.0 |
| Vitis vinifera (grape) seed oil | 6.0 |
| Glyceryl Stearate SE | 3.0 |
| Cetearyl alcohol | 2.0 |
| Dimethicone | 0.5 |
| Luvitol EHO^{c} (cetearyl octanoate) | 8.0 |
| Propylene glycol (and) BHT (and) ascorbyl palmitate (and) | 0.1 |
| glyceryl stearate (and) citric acid | |
| Phase B | |
| Propylene glycol | 3.0 |
| Glycerin 87% | 2.0 |
| Edeta BD^{c} (disodium EDTA) | 0.1 |
| D-Panthenol USP^{c} | 1.0 |
| Preservative | q.s. |
| Water | q.s. to 100 |
| Phase C | |
| Luvigel EM^{c} (caprylic/capric triglycerides (and) | 1.0 |
| sodium acrylates copolymer) | |
| Phase D | |
| Vitamin E Acetate^{c} | 0.5 |
| Perfume | q.s. |

^{a}Published at http://www.happi.com/current/Feb03formula.htm; Household and Personal Products Industry, 70 Hilltop Rd., Ramsey NJ 07446.
^{b}Procedure: Heat phase A and phase B to about 80° C. Stir phase B into phase A while homogenizing. Add phase C to phase A/B and homogenize again. Cool to about 40° C., add phase D and homogenize shortly. Comments: Viscosity: approx. 25,000 mPa * s (Brookfield); pH value: 6.5
^{c}BASF, 3000 Continental Drive North, Mount Olive, NJ 07828

TABLE IV
Natural Fluid Emulsion^{a,b}

| Ingredients: | % Wt. |
|---|---|
| Phase A | |
| Tefose 2561^{c} (PEG-6 stearate and ceteth-20 | 6.00 |
| andglyceryl stearate and steareth-20) | |
| Cetyl alcohol | 1.00 |
| Hydrogenated castor oil | 0.50 |
| DPPG^{c} (propylene glycol dipelargonate) | 5.00 |
| Dimethicone | 4.00 |
| Tocopheryl acetate | 0.50 |
| Preservative | q.s. |
| Phase B | |
| Kiwi Original Extract^{c} (actinidia chinensis fruit water) | 37.90 |
| Carbomer | 0.10 |
| Phase C | |
| Sodium hydroxide (10% sol.) 0.20 | 0.20 |
| Grape Original Extract^{c} (vitis vinifera fruit water) | 30.00 |
| Cyclomethicone (and) dimethiconol | 4.00 |
| Propylene glycol | 5.00 |
| Aluminum starch octenyl succinate | 5.00 |
| Gatuline A^{c} (pilewort extract) | 0.10 |
| Perfume | 0.20 |

^{a}Published at http://www.happi.com/current/Sept00formula.htm; Household and Personal Products Industry, 70 Hilltop Rd., Ramsey NJ 07446.
^{b}Procedure: Heat A and B at 75° C. With stirring, add B into A. At 40° C., add C ingredients in the order listed.
^{c}Gattefosse Corporation, 372 Kinderkamack Rd., Westwood, NJ 07675.

TABLE V
Emulsifier-free Moisturizing Cream Gel^{a,b}

| Ingredients: | % Wt. |
|---|---|
| Phase A | |
| Ceraphyl 494^{c} (isocetyl isostearate) | 6.00 |
| Protachem ISP^{c} (Promateen) (isostearyl palmitate) | 3.60 |
| Ceraphyl 368^{c} (ethyl hexyl palmitate) | 2.40 |
| Phase B | |
| Aristoflex AVC^{d} (ammonium acryloyldimethyltaurate/ | 1.00 |
| vp copolymer) | |
| Phase C | |
| Water | 78.70 |
| Polyglykol 400^{d} (PEG-8) | 7.00 |
| Nipaguard DMDMH^{d} (DMDM hydantoin) | 1.00 |
| Phase D | |
| Fragrance | 0.30 |

^{a}Published at http://www.happi.com/current/Mar02formula.htm; Household and Personal Products Industry, 70 Hilltop Rd., Ramsey NJ 07446.
^{b}Procedure: Combine ingredients of phase A and mix completely. Add phase B to phase A with moderate mixing. Slowly add phase C with moderate to high mixing. Mix until fully hydrated. Add phase D and continue mixing. Homogenize until uniform. Comments: Manufacturing at room temperature; light and fresh, with moisturizing effect; provides smooth silky skin feel; fast-absorbing, non-greasy, non-sticky.
^{c}International Specialty Products, 1361 Alps Rd., Wayne, NJ 07470.
^{d}Clariant Corp., 624 E. Catawba Ave., M. Holly, NC 28120.

TABLE VI
Liquid Foundation^{a,b}

| Ingredients: | % Wt. |
|---|---|
| Phase A | |
| Deionized water | 60.68 |
| Potassium hydroxide, 10% solution | 0.98 |
| Crillet 4 NF^{c} (polysorbate 80) | 0.10 |
| Phase B | |
| Titanium dioxide/talc, 80% | 0.10 |
| Talc | 3.76 |
| Yellow iron oxide/talc, 80% | 0.80 |
| Red iron oxide/talc, 80% | 0.38 |
| Black iron oxide/talc, 80% | 0.06 |
| Phase C | |
| Propylene glycol | 4.00 |
| Veegum Regular^{d} (magnesium aluminum silicate) | 1.00 |
| Phase D | |
| Propylene glycol | 2.00 |
| CMC 7H3SF^{e} (cellulose gum) | 0.12 |
| Phase E | |
| Cromollient DP3-A^{c} (di-PPG-3 myristyl ether adipate) | 12.00 |
| Crodafos CS 20 Acid^{c} (cetearyl alcohol (and) ceteth-20 | 3.00 |
| phosphate(and) dicetyl phosphate) | |
| Volpo S-10^{c} (steareth-10) | 2.00 |
| Crodacol C-70^{c} (cetyl alcohol) | 0.62 |
| Volpo S-2^{c} (steareth-2) | 0.50 |
| Phase F | |
| Germaben II^{f} (propylene glycol (and) diazolidinyl urea (and) | 1.00 |
| methylparaben (and) propylparaben) | |
| ^{a}Published at http://www.happi.com/current/Jul01formula.htm; Household and Personal Products Industry, 70 Hilltop Rd., Ramsey NJ 07446. | |
| ^{b}Procedure: Combine phase A ingredients and homogenize at low speed, heating to 75° C. Avoid aeration. Premill pigments of phase B in Osterizer for 3 × 15 seconds at high speed, weighing out 10% extra to compensate for loss. Slowly, add phase B while homogenizing on low speed and cover beaker. Combine phase C ingredients, mix to a slurry, then add to phase A/B. Continue to heat to 85-90° C. and maintain | |
| #temperature for 15 minutes. Cool to 75-80° C. Combine phase D ingredients and mix to a slurry, add to mixture and homogenize until uniform. Check beaker weight and add water to 20 grams over theoretical weight for a 1-kg batch. Cover beaker and bring temperature back up to 77-80° C. Combine ingredients of phase E and heat separately to 77-80° C. When both beakers reach the same temperature (77-80° C.), add | |
| #phase E to main beaker and maintain temperature for 15 minutes. Remove from heat and cool to 55° C. with gentle homogenization, allowing for 10% water loss. Cool to 45° C. in a water bath. Add phase F and cool to 30° C. Adjust pH to 7.5 with 25% NaOH solution. | |

^{c}Croda, Inc., 7 Century Dr., Parsippany, NJ 07054.
^{d}R. T. Vanderbilt Company, Inc., 30 Winfield St., Norwalk, CT 06856.
^{e}Hercules, Inc., Aqualon Division, 1313 N. Market St., Wilmington, DE 19894.
^{f}International Specialty Products, 1361 Alps Rd., Wayne, NJ 07470.

TABLE VII
Dry Oil Moisturizing Body Spray^{a,b}

| Ingredients: | % Wt. |
|---|---|
| Phase A | |
| Isododecane^{c} | 63.25 |
| Liponate PC^{d} (propylene glycol dicaprylate/dicaprate) | 11.00 |
| Crodamol PMP^{e} (PPG-2 myristyl ether propionate) | 9.00 |
| Liponate GC^{d} (caprylic/capric triglyceride) | 5.00 |
| High oleic safflower oil (Collaborative) | 3.00 |
| Isoeicosane^{c} | 2.00 |
| Ceraphyl 375^{f} (isostearyl neopentanoate) | 1.50 |
| Arlacel 186^{g} (glyceryl oleate (and) propylene glycol) | 1.50 |
| Propylparaben^{f} | 0.20 |
| BHT | 0.05 |
| Phase B | |
| Arlacel 186^{g} (glyceryl oleate (and) propylene glycol) | 3.00 |
| Fragrance | 0.50 |

^{a}Published at http://www.happi.com/current/Jul01formula.htm; Household and Personal Products Industry, 70 Hilltop Rd., Ramsey NJ 07446.
^{b}Procedure: Blend all of phase A ingredients in a suitable vessel. Mix until clear. Pre-mix phase B ingredients. Mix until clear. Add B to A and mix until clear. Pour into suitable containers.
^{c}Presperse Inc., 141 Ethel Road West, Piscataway, NJ 08854.
^{d}Lipo Chemicals, 207 19^{th} Ave., Paterson, NJ 07504.
^{e}Croda, Inc., 7 Century Dr., Parsippany, NJ 07054.
^{f}International Specialty Products, 1361 Alps Rd., Wayne, NJ 07470.
^{g}ICI India, Uniquema, 1000 Uniqema Blvd. New Castle DE 19720.

Suitable antioxidants include tocopheryl acetate, sodium ascorbyl phosphate, butylated hydroxyanisole, propylene glycol, propyl gallate, citric acid and others approved for topical or cosmetic use.

Suitable anti-inflammatory agents include but are not limited to d-panthenol, cucumber extract and others approved for topical or cosmetic use.

Suitable pH adjusting agents include but are not limited to triethanolamine and sodium hydroxide, with triethanolamine preferred.

In formulating the cabbage extract compositions of the present invention, the cabbage extract is preferably added to the composition after the water and oil phases are combined. Mixing the water and oil portions before adding the cabbage extract provides a safeguard against the possibility of other ingredients reacting unfavorably with the cabbage extract during the mixing process.

The cabbage extract compositions and methods of use of the present invention are further illustrated in detail in the examples provided below, but these examples are not to be construed to limit the scope of the invention in any way. While these examples describe the invention, it is understood that modifications to the compositions and methods are well within the skill of one in the art, and such modifications are considered within the scope of the invention.

### EXAMPLE 1

### Exemplary Cabbage Extract Composition

The following cabbage extract composition was used for treating women for the purposes of lactation cessation and relief of breast engorgement symptoms: 5% cabbage extract, 0.5% grape seed extract, and 0.5% cucumber extract in the enriched daily lotion presented in Table I (all percentages by weight of final formulation).

### EXAMPLE 2

### Postpartum Treatment with Cabbage Extract Composition for Lactation Cessation

A mother reported having previously experienced pain associated with breast engorgement postpartum following the birth of her first two children. Beginning on the day of delivery of her third child, the 31 year old postpartum mother began applying a cabbage extract composition given in Example 1 to the entire area of her breasts four times daily for three days and bound her breasts with a sports bra. The mother reported that her milk did not come in and that she experienced no pain associated with breast engorgement.

### EXAMPLE 3

### Postpartum Treatment with Cabbage Extract Composition for Lactation Cessation

A mother reported having previously experienced pain associated with breast engorgement postpartum following the birth of her first child. Beginning on the day of delivery of her second child, the 36 year old postpartum mother began applying the cabbage extract composition to the entire area of her breasts four times daily for three days and bound her breasts with a sports bra. The mother reported that her milk did not come in and that she experienced no pain associated with breast engorgement.

### EXAMPLE 4

### Post-breastfeeding Treatment with Cabbage Extract Composition for Lactation Cessation

A mother reported having previously experienced pain associated with breast engorgement during the weaning of her first child. After breastfeeding her second child for nine months, the 35 year old mother began applying the cabbage extract composition to the entire area of her breasts three to four times daily for three days and bound her breasts with a sports bra. The mother reported that her milk dried up in three days and that she experienced fullness but had no pain associated with breast engorgement compared to her experience during the weaning of her first child.

### EXAMPLE 5

### Post-breastfeeding Treatment with Cabbage Extract Composition for Lactation Cessation

After breastfeeding for three months, a 35 year old mother began applying the cabbage extract composition to the entire area of her breasts three to four times daily for three days and bound her breasts with a sports bra. The mother reported that her milk dried up in two days and that she experienced no pain associated with breast engorgement.

### EXAMPLE 6

### Postpartum Breastfeeding Treatment with Cabbage Extract Composition for Breast Engorgement

A 29 year old primigravida mother experienced intense breast engorgement on Day 4 after a successfull Caesarean section delivery. Although the mother wanted to breastfeed, the engorgement pain was too intense in both breasts to permit either breastfeeding or pumping of breastmilk. The cabbage extract composition was applied to the entire area of both breasts. Approximately thirty minutes later, the breast pain had decreased and the breast tissue had softened to permit breastfeeding. The mother applied the cabbage extract composition once more on the following day and continued breastfeeding thereafter without discomfort.

### REFERENCES CITED

American Academy of Pediatrics Work Group on Breastfeeding. 1997. “Breastfeeding and the use of human milk.” Pediatrics 100:1035-1039.

American Academy of Pediatrics, Committee on Pediatric Aids. 1995. “Human milk, breastfeeding, and transmission of human immunodeficiency virus in the United States,” Pediatrics 96:977-979.

American Academy of Pediatrics, Committee on Drugs. 1994. “The transfer of drugs and other chemicals into human milk,” Pediatrics 93:137-150.

Arnon S. 1984. “Breast feeding and toxigenic intestinal infections: missing links in crib death?” Rev Infect Dis 6:S193-S201

Beaudry, et al. 1995. “Relation between infant feeding and infections during the first six months of life,” J Pediatr 126:191-197.

Chua, et al. 1994. “Influence of breastfeeding and nipple stimulation on postpartum uterine activity,” Br J Obstet Gynaecol 101:804-805.

Cochi, et al. 1986. “Primary invasive Haemophilus influenzae type b disease: a population-based assessment of risk factors,” J Pediatr 108:887-896.

Corrieri, D. 1992. “Cabbage leaves: an effective treatment for swollen tissues,” J Hum Lact 8:126-127.

Cumming, R G. and Klineberg, R J. 1993. “Breastfeeding and other reproductive factors and the risk of hip fractures in elderly woman,” Int J Epidemiol 22:684-691.

Dewey, et al. 1993. “Maternal weight-loss patterns during prolonged lactation,” Am J Clin Nutr 58:162-166.

Dewey, et al. 1995. “Differences in morbidity between breast-fed and formula-fed infants,” J Pediatr 126:696-702.

Duncan, et al. 1993. “Exclusive breast-feeding for at least 4 months protects against otitis media,” Pediatrics; 91:867-872.

Ford, et al. 1993. “Breastfeeding and the risk of sudden infant death syndrome,” Int J Epidemiol 22:885-890.

Greco, et al. 1988. “Case control study on nutritional risk factors in celiac disease,” J Pediatr Gastroenterol Nutr 7:395-399.

Iffy, et al. 1998. “Severe cardiac dysrhythmia in patients using bromocriptine postpartum,” Am J Ther 5:111-115.

Kennedy, K I. and Visness C M. 1992. “Contraceptive efficacy of lactational amenorrhoea,” Lancet 339:227-230.

Koletzko, et al. 1989. “Role of infant feeding practices in development of Crohn's disease in childhood,” Br Med J 298:1617-1618.

Lucas A. and Cole T J. 1990. “Breast milk and neonatal necrotising enterocolitis,” Lancet 336:1519-1523.

Lucas, et al. 1990. “Early diet of preterm infants and development of allergic or atopic disease: randomised prospective study,” Br Med J 300:837-840.

Mayer, et al. 1988. “Reduced risk of IDDM among breast-fed children,” Diabetes 37:1625-1632.

Melton, et al. 1993. “Influence of breastfeeding and other reproductive factors on bone mass later in life,” Osteoporos Int 3:76-83.

Morrow-Tlucak, et al. 1988. “Breastfeeding and cognitive development in the first 2 years of life,” Soc Sci Med 26:635-639.

Newcomb, et al. 1994. “Lactation and a reduced risk of premenopausal breast cancer,” N Engl J Med 330:81-87.

Nicodem, et al. 1993. “Do cabbage leaves prevent breast engorgement? A randomized, controlled study,” Birth 20:61-64.

Pisacane, et al. 1992. “Breast-feeding and urinary tract infection,” J Pediatr 120:87-89.

Rigas, et al. 1993. “Breast-feeding and maternal smoking in the etiology of Crohn's disease and ulcerative colitis in childhood,” Ann Epidemiol 3:387-392.

Roberts, K L. 1995. “A comparison of chilled cabbage leaves and chilled gelpaks in reducing breast engorgement,” J Hum Lact 11:17-20.

Roberts, et al. 1995. “A comparison of chilled and room temperature cabbage leaves in treating breast engorgement,” J Hum Lact 11:191-194.

Roberts, et al. 1998. “Effects of cabbage leaf extract on breast engorgement,” J Hum Lact 14:231-236.

Rohr, et al. 1985. “Inborn errors of metabolism.” In: Walker W A. and Watkins J B., eds. Nutrition in Pediatrics, Boston, Mass.: Little, Brown; p. 412.

Rosenblatt, K A. and Thomas D B. 1993. “WHO collaborative study of neoplasia and steroid contraceptives,” Int J Epidemiol 22:192-197.

Rosier, W. 1988. “Cool cabbage compresses,” Breastfeed Rev 12:2-31.

Shu, et al. 1995. “Infant breastfeeding and the risk of childhood lymphoma and leukaemia,” Int J Epidemiol 24:27-32.

Snowden, et al. 2001. “Treatments for breast engorgement during lactation,” Cochrane Database Syst Rev 2CD000046.

Wright, et al. 1989. “Breast feeding and lower respiratory tract illness in the first year of life,” Br Med J 299:945-949

We claim:

1. A method for treating a postpartum woman to encourage the cessation of lactation, comprising applying a composition comprising an amount of greater than 1% cabbage leaf extract by weight of the final composition to the affected area of said woman's breasts repeatedly each day, commencing on the date of delivery and extending until lactation subsides.

2. The method of claim 1, wherein said composition comprises an amount of about 1.1% to about 25% cabbage leaf extract by weight of the final composition.

3. The method of claim 1, wherein said composition comprises 5% cabbage leaf extract by weight of the final composition.

4. The method of claim 1, wherein said composition comprises 5% cabbage leaf extract, 0.5% grape seed extract, and 0.5% cucumber extract by weight of the final composition.

5. The method of claim 1, 2, 3 or 4, further comprising the step of binding the breasts after said composition is applied to the breasts.

6. A method for treating a lactating woman to encourage the cessation of lactation, comprising applying a composition comprising an amount of greater than 1% cabbage leaf extract by weight of the final composition to the affected area of said woman's breasts repeatedly each day, commencing on a desired date and extending until lactation subsides.

7. The method of claim 6, wherein said composition comprises an amount of about 1.1% to about 25% cabbage leaf extract by weight of the final composition.

8. The method of claim 6, wherein said composition comprises 5% cabbage leaf extract by weight of the final composition.

9. The method of claim 6, wherein said composition comprises 5% cabbage leaf extract, 0.5% grape seed extract, and 0.5% cucumber extract by weight of the final composition.

10. The method of claim 6, 7, 8 or 9, further comprising binding the breasts after said composition is applied to the breasts.

11. A method for treating a lactating woman in need of treatment for or prevention of breast engorgement during lactation cessation, comprising applying a composition comprising an amount of greater than 1% cabbage leaf extract by weight of the final composition to the affected area of said woman's breasts repeatedly each day, commencing on a desired date and extending until lactation subsides.

12. The method of claim 11, wherein said composition comprises an amount of about 1.1% to about 25% cabbage leaf extract by weight of the final composition.

13. The method of claim 11, wherein said composition comprises 5% cabbage leaf extract by weight of the final composition.

14. The method of claim 11, wherein said composition comprises 5% cabbage leaf extract, 0.5% grape seed extract, and 0.5% cucumber extract by weight of the final composition.

15. The method of claim 11, 12, 13 or 14, further comprising binding the breasts after said composition is applied.

16. A method for treating a lactating woman for symptoms of breast engorgement, comprising applying one application of a composition comprising an amount of greater than 1% cabbage leaf extract by weight of the final composition to the affected area of said woman's breasts, commencing upon appearance of said symptoms.

17. The method of claim 16, wherein said composition comprises an amount of about 1.1% to about 25% cabbage leaf extract by weight of the final composition.

18. The method of claim 16, wherein said composition comprises 5% cabbage leaf extract by weight of the final composition.

19. The method of claim 16, wherein said composition comprises 5% cabbage leaf extract, 0.5% grape seed extract, and 0.5% cucumber extract by weight of the final composition.

20. The method of claim 16, 17, 18, or 19, further comprising applying said composition to the affected area of said woman's breasts once per day until symptoms subside.

21. A method for preventing symptoms of breast engorgement in a postpartum woman's breasts, comprising applying one application of a composition comprising an amount of greater than 1% cabbage leaf extract by weight of the final composition to said woman's breasts, commencing on the day milk comes in.

22. The method of claim 21, wherein said composition comprises an amount of about 1.1% to about 25% cabbage leaf extract by weight of the final composition.

23. The method of claim 21, wherein said composition comprises 5% cabbage leaf extract by weight of the final composition.

24. The method of claim 21, wherein said composition comprises 5% cabbage leaf extract, 0.5% grape seed extract, and 0.5% cucumber extract by weight of the final composition.

25. The method of claim 21, 22, 23 or 24, further comprising applying said composition to said woman's breasts once per day as symptoms appear.

26. A formulation for preventing or treating breast engorgement, comprising an extract of a vegetable leaf selected from the group consisting of Brassica oleracea capitata and Brassica Campestris Pekinenis in an amount comprising greater than 1% by weight of said formulation and an acceptable topical carrier.

27. The formulation of claim 26, wherein said formulation comprises an amount of about 1.1% to about 25% cabbage leaf extract by weight of the final formulation.

28. The formulation of claim 26, wherein said formulation comprises 5% cabbage leaf extract by weight of the final formulation.

29. The formulation of claim 26, wherein said formulation comprises 5% cabbage leaf extract, 0.5% grape seed extract, and 0.5% cucumber extract by weight of the final formulation.

30. A formulation for promoting lactation cessation in a lactating woman, comprising an extract of a vegetable leaf selected from the group consisting of Brassica oleracea capitata and Brassica Campestris Pekinenis in an amount comprising greater than 1% by weight of said formulation and an acceptable topical carrier.

31. The formulation of claim 30, wherein said formulation comprises an amount of about 1.1% to about 25% cabbage leaf extract by weight of the final formulation.

32. The formulation of claim 30, wherein said formulation comprises 5% cabbage leaf extract by weight of the final formulation.

33. The formulation of claim 30, wherein said formulation comprises 5% cabbage leaf extract, 0.5% grape seed extract, and 0.5% cucumber extract by weight of the final formulation.

34. A kit for the treatment of breast engorgement, comprising a formulation consisting of a cabbage extract and an acceptable topical carrier, said cabbage extract comprising greater than 1% by weight of said formulation, and instructions for the application of said formulation to a lactating woman.

35. The kit of claim 34, further comprising a breast-binding means selected from the group consisting of a tight-fitting garment or bandaging material.

36. A kit for promoting lactation cessation in a lactating woman, comprising a formulation consisting of a cabbage extract and an acceptable topical carrier, said cabbage extract comprising greater than 1% by weight of said formulation, and instructions for the application of said formulation to said lactating woman.

37. The kit of claim 36, further comprising a breast-binding means selected from the group consisting of a tight-fitting garment or bandaging material.
